(19) United States
(12) Patent Application Publication
(10) Pub. No.: US 2025/0369943 A1
(43) Pub. Date: Dec. 4, 2025

(54) CONTROLLING VOLATILE ORGANIC COMPOUND OFF-GASSING IN A DATACENTER

(71) Applicant: INTERNATIONAL BUSINESS MACHINES CORPORATION, ARMONK, NY (US)

(72) Inventors: REBECCA MORONES, BERTHOUD, CO (US); JOHN S. WERNER, FISHKILL, NY (US); ARKADIY O. TSFASMAN, WAPPINGERS FALLS, NY (US)

(21) Appl. No.: 18/679,662

(22) Filed: May 31, 2024

(51) Int. Cl. G01N 33/00

(52) U.S. Cl. CPC G01N 33/0075

(57) ABSTRACT

Methods, apparatus, systems, and computer program products for controlling VOC (Volatile Organic Compound) off-gassing in a datacenter include receiving sensor data from a plurality of VOC sensors in the datacenter; and controlling off-gassing in the datacenter based on the sensor data and human presence within the datacenter.

### BACKGROUND

The present disclosure relates to methods, apparatus, and products for controlling VOC (Volatile Organic Compound) off-gassing in a datacenter.

### SUMMARY

According to embodiments of the present disclosure, various methods, apparatus, and products for controlling VOC off-gassing in a datacenter are described herein. In some aspects, controlling VOC off-gassing in a datacenter includes receiving sensor data from a plurality of VOC sensors in the datacenter and controlling off-gassing in the datacenter based on the sensor data and human presence within the datacenter.

### BRIEF DESCRIPTION OF THE DRAWINGS

FIG. 1 sets forth a network diagram of an example system configured for controlling off-gassing of VOCs in a datacenter according to various embodiments of the present disclosure.

FIG. 2 sets forth an example bird-eye view of a datacenter configured for controlled off-gassing of VOCs according to various embodiments of the present disclosure.

FIG. 3 sets forth a flow chart illustrating an example method of controlling VOC off-gassing in a datacenter in accordance with embodiments of the present disclosure.

FIG. 4 sets forth a flow chart illustrating another example method of controlling VOC off-gassing in a datacenter that is based in on detection of human presence in accordance with embodiments of the present disclosure.

FIG. 5 sets forth a flow chart illustrating another example method of controlling VOC off-gassing in a datacenter in which a safe route through a datacenter is generated in accordance with embodiments of the present disclosure.

FIG. 6 sets forth a flow chart illustrating another example method of controlling VOC off-gassing in a datacenter in which a representation of VOC concentrations is generated in accordance with embodiments of the present disclosure.

FIG. 7 sets forth an example computing environment according to aspects of the present disclosure.

### DETAILED DESCRIPTION

Volatile organic compounds or ‘VOCs’ are gases that are emitted into the air from products or processes. VOCs have a high vapor pressure and low water solubility. Many VOCs are human-made chemicals that are used and produced in the manufacture of paints, pharmaceuticals, and refrigerants. VOCs typically are industrial solvents, such as trichloroethylene; fuel oxygenates, such as methyl tert-butyl ether (MTBE); or by-products produced by chlorination in water treatment, such as chloroform. VOCs are often components of petroleum fuels, hydraulic fluids, paint thinners, and dry-cleaning agents. VOCs are common ground-water contaminants.

Volatile organic compounds (VOCs) are emitted as gases from certain solids or liquids. VOCs include a variety of chemicals, some of which may have short- and long-term adverse health effects. Concentrations of many VOCs are consistently higher indoors (up to ten times higher) than outdoors.

Breathing VOCs can irritate the eyes, nose, and throat, can cause difficulty breathing and nausea, and can damage the central nervous system and other organs. Some VOCs can cause cancer. Some VOCs are harmful by themselves, while others can react with other gases and form other air pollutants.

Examples of VOCs include benzene, ethylene glycol, formaldehyde, methylene chloride, tetrachloroethylene, acetaldehyde, toluene, xylene, and 1,3-butadiene. Computer and electronic components of servers, networking equipment, storage devices, rack units, and the like in a datacenter may include VOCs, especially when the components are relatively new manufactures. Over time, components naturally generate less VOC gas. Additionally, due to the high number of such components and the enclosed nature of a datacenter, the risk of harmful or dangerous levels of VOCs is higher in a datacenter than other locations. When maintenance, installation, or inspections are carried out by human users, VOCs in a datacenter can present a potential threat.

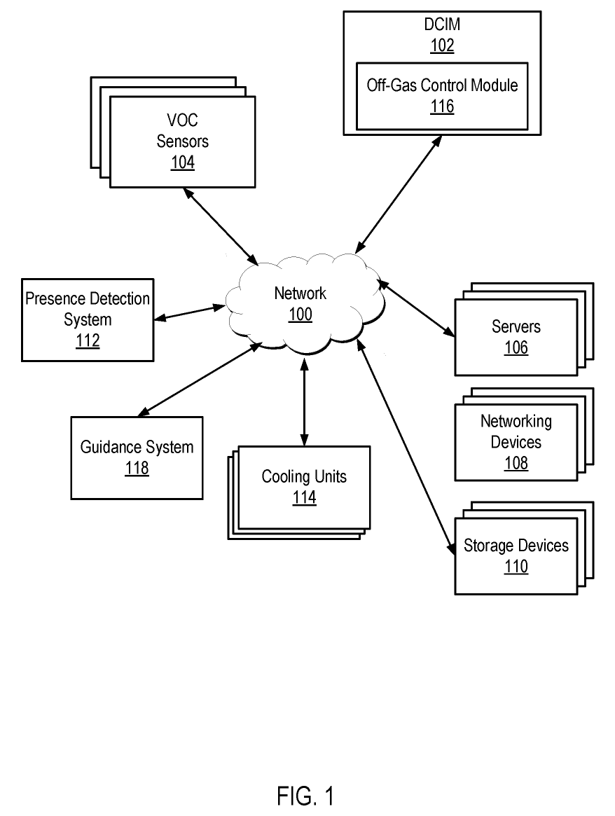

FIG. 1 sets forth a network diagram of an example system configured for controlling off-gassing of VOCs in a datacenter according to various embodiments of the present disclosure. The example system of FIG. 1 includes servers 106, networking devices 108, storage devices 110, VOC sensors 104, a DCIM (Datacenter infrastructure manager), a presence detection system 112, cooling units 114, and a guidance system 118. The components of the system are coupled for data communications through a network 100. Such a network may be wired, wireless, or some combination of the two. The network 100 in the example of FIG. 1 may be formed of a LAN (Local Area Network), a WAN (Wide Area Network), a SAN (Storage Area Network) or any combination of such networks and others as will occur to those of skill in the art.

The servers 106, networking device 108, and storage devices 110 may be housed in rack units in a datacenter. The servers 106 may include components such as network interfaces, processors, graphics processors, accelerators, FPGAs, ASICs, computer memory in the form of DRAM or the like, storage devices in the form of hard disc drives or SSDs (Solid State Drives), and other components. The networking devices 108 may include switches, routers, firewalls, and the like. The storage devices 110 may include arrays of storage drives (HDDs or SSDs or a combination).

The DCIM in the example of FIG. 1 may, like the servers 106, networking devices 108 and storage devices 110, also be housed in a rack unit inside the datacenter. In some embodiments, however, the DCIM may be housed in a location that is remote to the datacenter and coupled to the components of the datacenter via the network 100. The DCIM 102 is a computer system that includes a processor and computer memory coupled to the processor. Stored within the computer memory is a module of computer program instructions in the form of an off-gas control module 116. The off-gas control module 116 is executed to control off-gassing in the datacenter according to embodiments of the present disclosure.

More specifically, the off-gas control module 116 is configured to receive sensor data from a plurality of VOC sensors 104 in the datacenter and control off-gassing in the datacenter based on the sensor data and human presence within the datacenter. The VOC sensors 104 may be located throughout the datacenter at a variety of locations. For example, VOC sensors may be placed within a rack, within a server, within or near networking devices, within or near storage devices, at various locations within a floor or ceiling structure of the datacenter, or at other locations as will occur to those of skill in the art. VOC sensors monitor and quantify the level of concentration of a VOC within an area near the sensor. Different sensors may be configured to monitor different types of VOCs. For example, some sensors may be configured to measure gaseous formaldehyde particulate concentrations, while other sensors may be configured to measure gaseous toluene particulate concentrations. In some embodiments, VOC sensors are placed in locations known to be likely to generate VOCs of a type for which the sensor is configured to measure. Some VOC sensors may be installed by datacenter personnel after or in tandem with the installation of a rack unit, server, networking device, storage device, or other datacenter component. Some VOC sensors may be installed by the manufacturer of a rack unit, server, networking device, storage device, or other datacenter components prior to installation in the datacenter. In such an embodiment, the datacenter component within which the VOC sensor is installed may provide an interface for accessing the VOC sensor through a variety of means including, in-band network communications, exposing an API (Application Programming Interface), out-of-band communications such as I2C or the like, wireless communications such as Near-Field communications (NFC) or Bluetooth, and so on.

The VOC sensors 104 communicate the measured readings to the off-gas control module 116. The off-gas control module 116 may periodically poll the sensors 104 for data, the sensors 104 may periodically submit data to the off-gas control module, or the off-gas control module may query the sensors on-demand for sensor data. The data may include a location of the VOC sensor or may include an identifier of the VOC sensor from which a location in the datacenter can be derived. For example, VOC sensors may register with the off-gas control module and report back an identifier of the sensor itself along with a component identifier or a two- or three-dimensional coordinate that maps to a physical location within the datacenter. An administrator may associate a location within the datacenter with the VOC sensor identifier such that data received from the sensor can be associated with a particular location in the datacenter. Such a location can be expressed in a variety of ways, such as a rack unit identifier, a drawer identifier, a server identifier, an X, Y, Z coordinate, or the like.

Once data is received from the VOC sensors 104, the off-gas control module 116 controls off-gassing of VOCs in the datacenter based on the sensor data. The off-gas control module 116 may control the off-gassing of VOCs by increasing or decreasing cooling of components. As mentioned above, a component’s level of off-gassing of VOCs decreases over time. One way to expedite the timeline such that typical operation of a component results in acceptable levels of VOC off-gassing is to increase the temperature of that component. Increasing the temperature of a component will increase the amount of VOC off-gas, but will also shorten the overall time that dangerous or unacceptable levels of off-gassing occurs. Decreasing a component’s temperature, by contrast, results in a short-term reduction of off-gassing but increases the overall timeline during which the component may off-gas VOCs at unacceptable levels. To that end, the off-gas control module 116 may, for example, accelerate off-gassing of VOCs in the datacenter in areas of VOC off-gassing as indicated by the sensor data, by decreasing cooling of components within those areas of relatively high levels of VOCs. The off-gas control module 116 may control operation of the cooling units 114, decreasing cooling, to target the locations within the datacenter at which the sensor data indicates levels of VOCs above a predetermined threshold (levels at which VOC off-gassing is present). While the cooling units 114 may be operated to decrease cooling of components at those locations, thereby causing a rise in temperature of the locations, readers will understand that, in most cases, the cooling should not be decreased to the point at which a component’s temperature results in failure or a fault.

The off-gas control module 116 may also control off-gassing of VOCs in the datacenter by increasing cooling of components at one or more locations, thereby reducing VOC off gassing by those components temporarily. Such a temporary reduction of VOC off-gassing may be carried out when human presence is expected or otherwise detected by the presence detection system 112. In some embodiments, maintenance schedules, installation schedules, or system faults may be reviewed to identify times at which human presence is planned within the datacenter. In such instances, the off-gas control module 116 may use the sensor data to ensure that components exhibiting relatively high levels of VOC are cooled and the level of VOC is reduced below a threshold (sometimes referred to herein as a ‘safety threshold) prior to the scheduled human presence. In some instances, the off-gas control module may use the sensor data and the maintenance schedule to determine an intended location of human presence within the datacenter (such as a server scheduled for maintenance) and control the cooling in the datacenter to generate a ‘safe’ route through the datacenter where a human can reach the intended location without entering any areas of relatively high levels of VOC off-gassing. In such an embodiment, the off-gas control module 116 generally increases cooling of components showing levels of VOC off-gassing along the safe route.

In some embodiments, the presence detection system 112 of FIG. 1 is configured to detect presence of a humans within the datacenter and provide the location of such humans to the off-gas control module 116 in real time (or near-real-time). The off-gas control module 116, with knowledge of the present location of a user in the datacenter, can increase cooling to components near the user to decrease the off-gassing of VOCs for those systems which the sensor data indicates high VOC off-gassing. Additionally, in some embodiments, the off-gas control module may highlight locations or areas within the datacenter that presently have levels of off-gassing higher than a predetermined threshold through use of the guidance system 118 which may control various lighting throughout the datacenter. In some embodiments, the off-gas control module may highlight locations different (green lighting, orange lighting, or red lighting) based on the level of VOC off-gassing and a variety of ranges of off-gassing levels. Locations in the datacenter within a safe range of VOC levels may be highlighted in green lighting, for example, while locations having VOC levels in a medium range may be highlighted in yellow lighting, and locations having levels of VOC higher than a medium range may be highlighted in red lighting. Such a color scheme or highlighting provides a user with a real-time or near-real time indication of the levels of VOC off-gassing while the user is present within the datacenter.

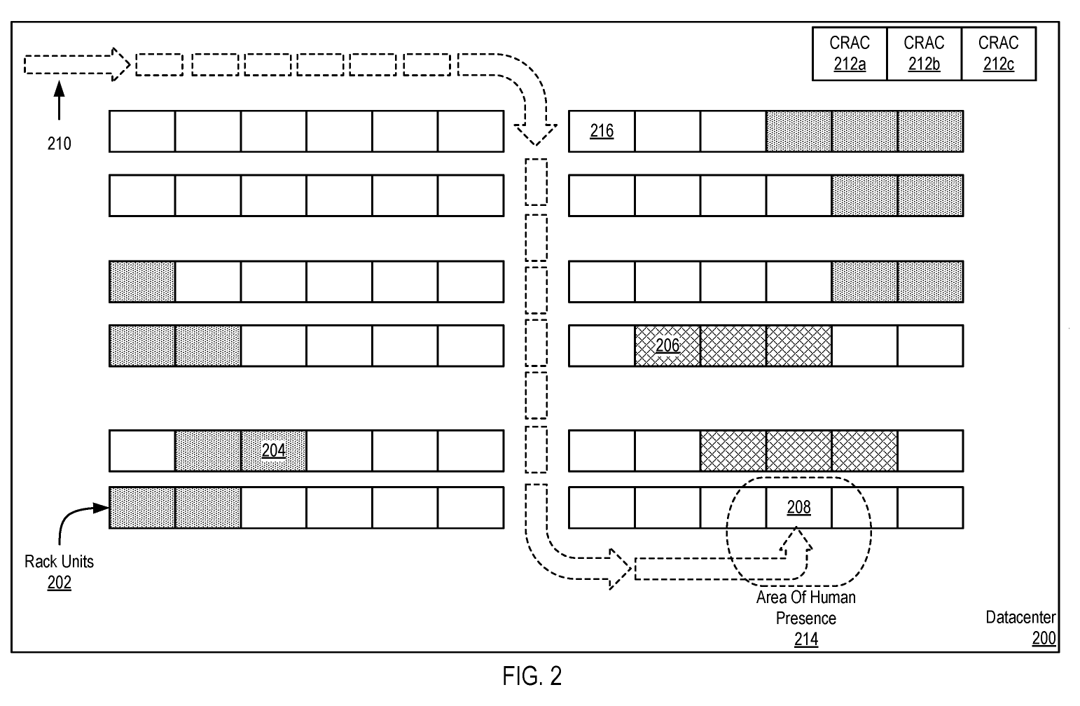

For further explanation, FIG. 2 sets forth an example bird-eye view of a datacenter 200 configured for controlled off-gassing of VOCs according to various embodiments of the present disclosure. The example datacenter 200 includes a number of rack units 202 arranged in aisles. Each of the rack units 202 may include any combination of servers, storage devices, networking devices, power supplies, power distribution units, input/output cards, and the like.

The example of FIG. 2 datacenter of FIG. 2 also includes several CRAC (Computer Room Air Conditioning) units 212a, 212b, 212c which can be utilized to adjust cooling throughout the datacenter.

One of the rack units in the example of FIG. 2 houses a DCIM as described above which executes an off-gas control module 216. Additionally, any number of the rack units 202 or any of the components within the rack units may include one or more VOC sensors like those described above. The floor or ceiling of the datacenter may also include VOC sensors.

To that end, in the example of FIG. 2, the VOC sensor data, received by the off-gas control module 216 indicates areas of high levels of VOC concentration 204, 206 (. In an example in which a user is scheduled to perform maintenance on a particular rack unit 208, the off-gas control module 216 may utilize the intended destination as well as the sensor data to increase cooling (through use of the CRAC units 212a, 212b, 212c) to some of the areas of higher levels of VOC concentration 206. In doing so, the VOC off-gassing will be decreased at these areas 206. More specifically, the off-gas control module 216 controls the cooling to ensure that the intended destination 208 of the human user is in an area that is safe (has relatively low levels of VOC off-gassing during the user’s presence in the datacenter). The area 214 of human presence, for example, near the intended destination (rack unit 208), is shown to have no or relatively little VOC off-gassing.

Additionally, in some embodiments, the off-gas control module 216 generates a path 210 for the human user to enter the datacenter and proceed to the intended destination 208. That path is generated such that a user following the path does not encounter an area of high level of VOC off-gassing. The path can be generated as a result of the sensor data without any control of off-gassing, or the off-gas control module can control the off-gassing in the datacenter to generate a safe path 210.

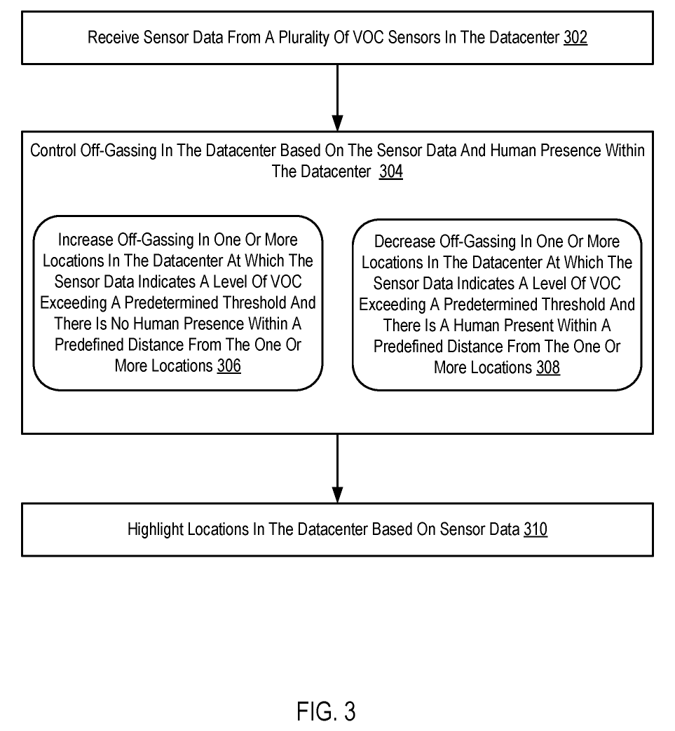

FIG. 3 sets forth a flow chart illustrating an example method of controlling VOC off-gassing in a datacenter in accordance with embodiments of the present disclosure. The method of FIG. 3 may be carried out by an off-gas control module such as that described above. Such an off-gas control module may be implemented with hardware, software, or some combination of the two. The method of FIG. 3 includes receiving 302 sensor data from a plurality of VOC sensors in the datacenter. The off-gas control module receives 302 sensor data through a variety of means including: in-band network communications, out-of-band network communications, wirelessly, or through wired means. The sensors, as mentioned above, can be placed at any location within the datacenter. The sensor data is associated with such a location. The location of a particular sensor in the datacenter may be input manually by a system administrator, may be received directly from the sensor itself, may be inferred through an identifier of the sensor or a system (such as rack unit or server) within which or near which the sensor is installed, and in other ways as will occur to readers of skill in the art.

The method of FIG. 3 also includes controlling 304 off-gassing in the datacenter based on the sensor data and human presence within the datacenter. In some embodiments, the off-gas control module may be configured to increase the rate of off-gassing of VOCs at locations in the datacenter at which the sensor data indicates a VOC off-gassing. Increasing the rate of VOC off-gassing at these locations, results in a shorter period of time during which components at these locations will continue to off-gas VOCs. As such, the off-gas control module in the example of FIG. 3 is also configured to increase such off-gassing only when there is no human presence in those locations.

When there is human presence (or there is expected to be human presence), the off-gas control module may be configured to decrease the rate of off-gassing of VOCs at locations in the datacenter at which the sensor data indicates a harmful concentration of VOCs than other locations. In such embodiments, decreasing the rate of VOC off-gassing at these locations results in a short-term reduction in VOC concentration (an immediate or on-demand reduction of harmful VOC gassing).

The off-gas control module may be configured as described above to increase off-gassing, decrease off-gassing, or a combination of the two. That is, the off-gas control module in some embodiments may be configured: only for increasing VOC off-gassing when no human is present at the locations in the datacenter having indicating VOCs off-gassing; only for decreasing VOC off-gassing when a human is present (or expected to be present) at the locations in the datacenter having a harmful concentration of VOCs; or both increasing VOC off-gassing when no human is present at the locations in the datacenter having VOCs off-gassing and decreasing VOC off-gassing when a human is present (or expected to be present) at the locations in the datacenter having a harmful concentration of VOCs.

In a larger datacenter having a significant amount of floorspace, VOC off-gassing can be increased in one area and decreased in another at the same time. That is, in a location of a large datacenter where no users are present and are not within a given area of that location, the off-gas control module may increase off-gassing at the location while in another area of the same datacenter a user is present, and the VOC off-gas control module reduces off-gassing in an area near the user.

To that end, controlling 304 off-gassing in the datacenter includes two optional steps of increasing 306 off-gassing in one or more locations in the datacenter at which the sensor data indicates a level of VOC exceeding a first predetermined threshold (indicating any VOC off-gassing) and there is no human presence within a predefined distance from the one or more locations and decreasing 308 off-gassing in one or more locations in the datacenter at which the sensor data indicates a level of VOC exceeding a second predetermined threshold (representing a harmful concentration of VOC off-gassing) and there is a human present within a predefined distance from the one or more locations. Additionally, each different sensor or different type of VOC may be associated with a different threshold.

Increasing 306 off-gassing can be carried out by reducing cooling to the locations in the datacenter at which the sensor data indicates a level of VOC that exceeds the first predetermined threshold. The amount of reduced cooling can be controlled such that the components being cooled do not exceed unsafe temperatures or temperatures that might cause component failure or unacceptable performance degradation. Decreasing 308 off-gassing can be carried out by increasing cooling to the locations in the datacenter having sensor data indicating a level of VOCs that exceed a second predetermined threshold. Cooling in a datacenter can be controlled in a variety of ways. For example, CRAC units can be controlled to increase or decrease cooling, speed of fans within servers or within racks, can be modified to control cooling of components, and so on.

The method of FIG. 3 also includes highlighting 310 locations in the datacenter based on the sensor data. The off-gas control module can highlight 310 locations in the datacenter in a manner to represent the location’s current level of off-gassing. Locations with high level of VOCs, for example, may be highlighted with a red light, areas of medium level of VOC may be highlighted with a yellow light, and areas of low level of VOC may be highlighted with a green light. In this manner, a user present in the datacenter can visually identify the current levels of VOC off-gassing at various locations in the datacenter. In another embodiment, the various off-gassing zones may be indicated on a mobile device of a user. In some embodiments, where extreme caution is desired, physical access to areas can be controlled via a badge reading, biometric scanner or gates/doors in different areas that do not open until VOCs have lowered to safe levels.

While shown in the example of FIG. 3 as a part of the method that also includes receiving 302 sensor data and controlling 304 off-gassing, readers will understand that highlighting 310 locations within a datacenter based on VOC sensor data may be carried out in embodiments that do not also control 304 off-gassing. That is, in some embodiments, an off-gas control module or another module may receive 302 VOC sensor data and highlight 310 locations in the datacenter according to or based on the received sensor data, without any control of off-gassing. The highlighting can be thought of as a ‘heatmap’ of sorts that visually depicts areas of differing levels of VOC off-gas concentrations. Such a VOC heatmap may be updated regularly, periodically, or on-demand. In some embodiments, for example, an off-gas control module or another module may, at regular intervals, receive VOC sensor data and highlight locations within the datacenter based on the sensor data.

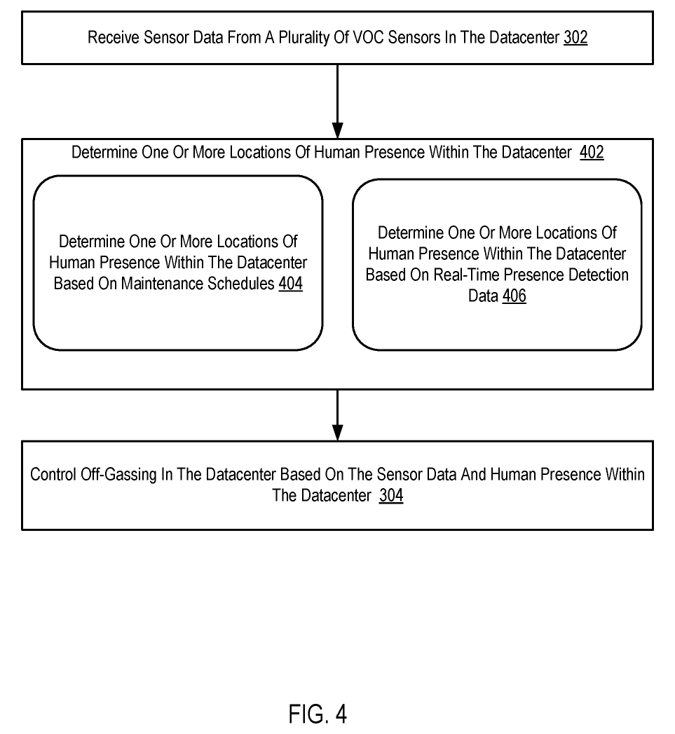

For further explanation, FIG. 4 sets forth a flow chart illustrating another example method of controlling VOC off-gassing in a datacenter in accordance with embodiments of the present disclosure. The method of FIG. 4 is similar to the method of FIG. 3 in that it also includes receiving 302 sensor data and controlling 304 off-gassing as described above. However, in the method of FIG. 4, such controlled VOC off-gassing is based on detection of human presence through one more means. More specifically, the method of FIG. 4 includes determining 402 one or more locations of human presence within the datacenter. Such a determination 402, in the method of FIG. 4, can be carried out by determining 404 one or more locations of human presence within the datacenter based on maintenance schedules. Such a determination 402, in the method of FIG. 4, can also be carried out by determining 406 one or more locations of human presence within the datacenter based on real-time presence detection data.

Determining 404 a location of human presence based on a maintenance schedule can be carried out by the off-gas control module described requesting maintenance schedules from a datacenter management system, with an API call to an API exposed for granting access to a repository of maintenance schedules, and in other ways. Such maintenance schedules may include intended destinations within the datacenter at which maintenance is to be performed (such as server identifier, rack unit number, component identifier, etc.), a number of and identifications of users to be performing the maintenance, and a date, time, and expected length of time of the maintenance to be performed. From such information, the off-gas control module can derive that a human will be present at a location within the datacenter, at a particular time or during a particular period of time. From that information, the off-gas control module may, before, during, and after the predefined time of maintenance, select locations to decrease off-gassing of VOCs (locations where human presence is to be expected) or increase off-gassing of VOCs (locations where human presence is not expected).

As another option, the off-gas control module can, in accordance with the method of FIG. 4, determine 406 one or more locations of human presence within the datacenter based on real-time (or near-real-time) presence detection data. Such real-time data can be from a variety of different types of sensors that may be located throughout the datacenter. Examples of sensors that can detect a human presence or from which human presence can be inferred include, proximity sensors, motion sensors, infra-red sensors, a live video stream, near-field communication (NFC) tags (where a user may utilize an NFC reader/tag to ‘check in’ to a particular location within the datacenter), door or other security clearings that require a security fob or security badge to access, Bluetooth, Lidar (Light Detection and Ranging), Radar (Radio Detection and Ranging), Sonar (sound navigation and ranging), and the like. Once presence is detected in real time through use of a presence detection sensor, the off-gas control module can, using the VOC sensor, control the off-gassing in the datacenter to decrease off-gassing of VOCs at any location near the human that is present within the datacenter. In some embodiments, with the knowledge of the user’s location within the datacenter, the off-gas control module may also increase VOC off-gassing at locations in the datacenter where the human is not present.

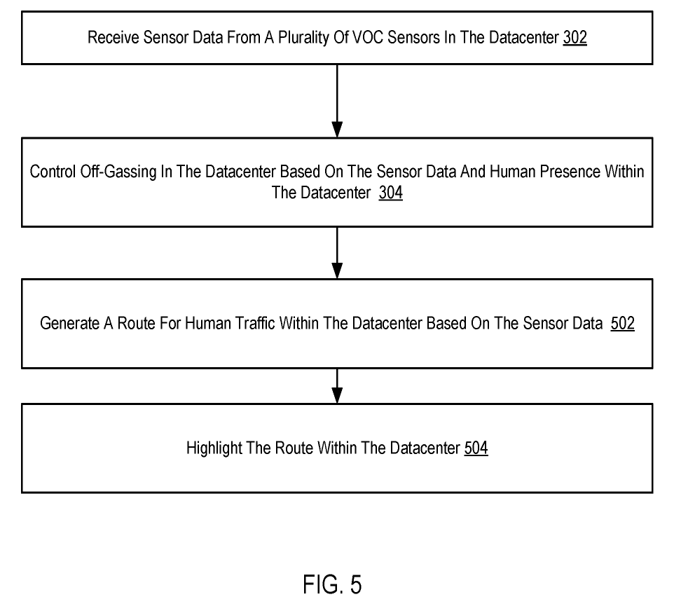

For further explanation, FIG. 5 sets forth a flow chart illustrating another example method of controlling VOC off-gassing in a datacenter in which a safe route through a datacenter is generated in accordance with embodiments of the present disclosure. The method of FIG. 5 is similar to those of the methods described above. However, the method of FIG. 5 includes generating 502 a route for human traffic within the datacenter based on the sensor data and highlighting 504 the route within the datacenter.

Such a route can be considered a ‘safe’ route for human traffic through the datacenter. For example, with knowledge of a user’s presence in the datacenter or more specifically the user’s intended destination within the datacenter (from a maintenance schedule, scheduled installation, and/or system faults/errors, for example), the off-gas control module may identify a route within the datacenter that the human can proceed and ensure that any locations along that route have reduced or eliminated levels of VOC off-gassing. In some embodiments, areas not on the route could have increased off-gassing.

The off-gas control module may also highlight 504 the route within the datacenter so a user can visibly or audibly follow the route. For example, the off-gas control module may activate designated lights in the ceiling or floor of the datacenter, or may produce audible sounds or verbal instructions to guide a user on the route.

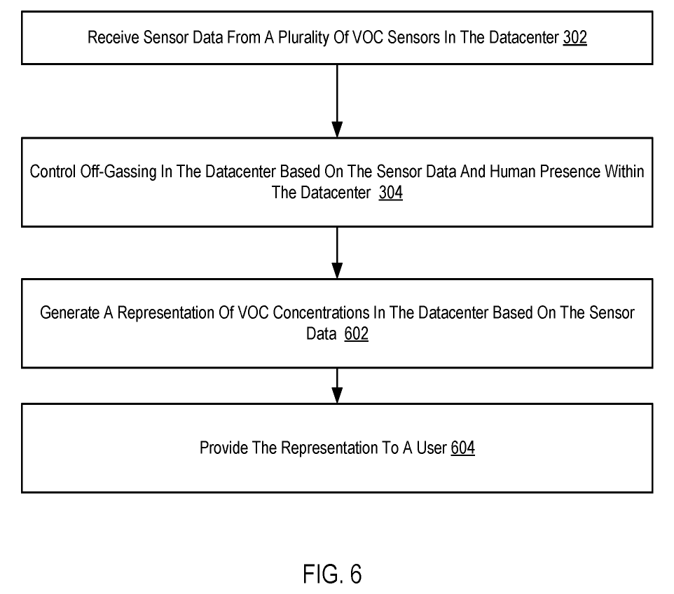

For further explanation, FIG. 6 sets forth a flow chart illustrating another example method of controlling VOC off-gassing in a datacenter in which a representation of VOC concentrations is generated in accordance with embodiments of the present disclosure. The method of FIG. 6 is similar to those described above. However, the method of FIG. 6 also includes generating 602 a representation of VOC concentrations in the datacenter based on the sensor data and providing 604 the representation to a user. The representation may be a graphical representation that can be accessible through a browser or other application. A user that intends to access the datacenter, for example, may request a current map of VOC off-gassing levels from the off-gas control module and the off-gas control module may provide a graphical representation of the current levels of VOC off-gassing to the user. Such a representation may be static – a snapshot taken at a particular time – or may be dynamic, changing regularly over time along with changes in VOC concentration at various locations within the datacenter. Additionally, the representation may include a ‘safe route’ as described above that directs the user on a path where VOC concentrations are relatively low or non-existent. Such a safe route may be generated upon request from the user or automatically without any request.

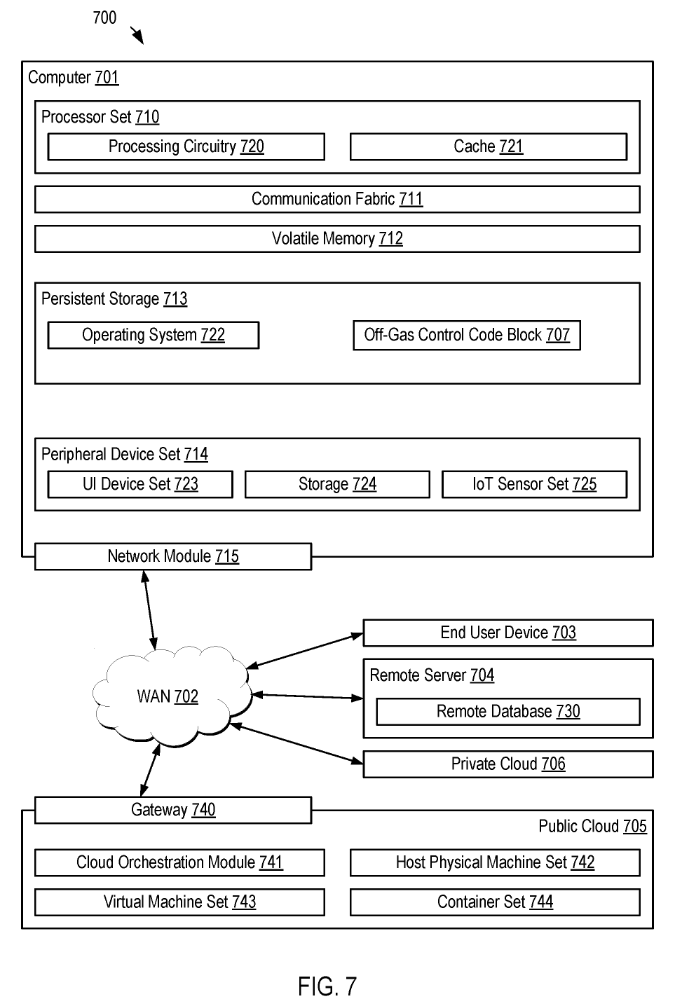

FIG. 7 sets forth an example computing environment according to aspects of the present disclosure. Computing environment 700 contains an example of an environment for the execution of at least some of the computer code involved in performing the various methods described herein, such as off-gas control code block 707. In addition to block 707, computing environment 700 includes, for example, computer 701, wide area network (WAN) 702, end user device (EUD) 703, remote server 704, public cloud 705, and private cloud 706. In this embodiment, computer 701 includes processor set 710 (including processing circuitry 720 and cache 721), communication fabric 711, volatile memory 712, persistent storage 713 (including operating system 722 and block 707, as identified above), peripheral device set 714 (including user interface (UI) device set 723, storage 724, and Internet of Things (IoT) sensor set 725), and network module 715. Remote server 704 includes remote database 730. Public cloud 705 includes gateway 740, cloud orchestration module 741, host physical machine set 742, virtual machine set 743, and container set 744.

Computer 701 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 730. As is well understood in the art of computer technology, and depending upon the technology, performance of a computer-implemented method may be distributed among multiple computers and/or between multiple locations. On the other hand, in this presentation of computing environment 700, detailed discussion is focused on a single computer, specifically computer 701, to keep the presentation as simple as possible. Computer 701 may be located in a cloud, even though it is not shown in a cloud in FIG. 7. On the other hand, computer 701 is not required to be in a cloud except to any extent as may be affirmatively indicated.

Processor set 710 includes one, or more, computer processors of any type now known or to be developed in the future. Processing circuitry 720 may be distributed over multiple packages, for example, multiple, coordinated integrated circuit chips. Processing circuitry 720 may implement multiple processor threads and/or multiple processor cores. Cache 721 is memory that is located in the processor chip package(s) and is typically used for data or code that should be available for rapid access by the threads or cores running on processor set 710. Cache memories are typically organized into multiple levels depending upon relative proximity to the processing circuitry. Alternatively, some, or all, of the cache for the processor set may be located “off chip.” In some computing environments, processor set 710 may be designed for working with qubits and performing quantum computing.

Computer readable program instructions are typically loaded onto computer 701 to cause a series of operational steps to be performed by processor set 710 of computer 701 and thereby effect a computer-implemented method, such that the instructions thus executed will instantiate the methods specified in flowcharts and/or narrative descriptions of computer-implemented methods included in this document. These computer readable program instructions are stored in various types of computer readable storage media, such as cache 721 and the other storage media discussed below. The program instructions, and associated data, are accessed by processor set 710 to control and direct performance of the computer-implemented methods. In computing environment 700, at least some of the instructions for performing the computer-implemented methods may be stored in block 707 in persistent storage 713.

Communication fabric 711 is the signal conduction path that allows the various components of computer 701 to communicate with each other. Typically, this fabric is made of switches and electrically conductive paths, such as the switches and electrically conductive paths that make up buses, bridges, physical input / output ports and the like. Other types of signal communication paths may be used, such as fiber optic communication paths and/or wireless communication paths.

Volatile memory 712 is any type of volatile memory now known or to be developed in the future. Examples include dynamic type random access memory (RAM) or static type RAM. Typically, volatile memory 712 is characterized by random access, but this is not required unless affirmatively indicated. In computer 701, the volatile memory 712 is located in a single package and is internal to computer 701, but, alternatively or additionally, the volatile memory may be distributed over multiple packages and/or located externally with respect to computer 701.

Persistent storage 713 is any form of non-volatile storage for computers that is now known or to be developed in the future. The non-volatility of this storage means that the stored data is maintained regardless of whether power is being supplied to computer 701 and/or directly to persistent storage 713. Persistent storage 713 may be a read only memory (ROM), but typically at least a portion of the persistent storage allows writing of data, deletion of data and re-writing of data. Some familiar forms of persistent storage include magnetic disks and solid-state storage devices. Operating system 722 may take several forms, such as various known proprietary operating systems or open-source Portable Operating System Interface-type operating systems that employ a kernel. The code included in block 707 typically includes at least some of the computer code involved in performing the computer-implemented methods described herein.

Peripheral device set 714 includes the set of peripheral devices of computer 701. Data communication connections between the peripheral devices and the other components of computer 701 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 723 may include components such as a display screen, speaker, microphone, wearable devices (such as goggles and smart watches), keyboard, mouse, printer, touchpad, game controllers, and haptic devices. Storage 724 is external storage, such as an external hard drive, or insertable storage, such as an SD card. Storage 724 may be persistent and/or volatile. In some embodiments, storage 724 may take the form of a quantum computing storage device for storing data in the form of qubits. In embodiments where computer 701 is required to have a large amount of storage (for example, where computer 701 locally stores and manages a large database), this storage may be provided by peripheral storage devices designed for storing very large amounts of data, such as a storage area network (SAN) that is shared by multiple, geographically distributed computers. IoT sensor set 725 is made up of sensors that can be used in Internet of Things applications. For example, one sensor may be a thermometer and another sensor may be a motion detector.

Network module 715 is the collection of computer software, hardware, and firmware that allows computer 701 to communicate with other computers through WAN 702. Network module 715 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 715 are performed on the same physical hardware device. In other embodiments (for example, embodiments that utilize software-defined networking (SDN)), the control functions and the forwarding functions of network module 715 are performed on physically separate devices, such that the control functions manage several different network hardware devices. Computer readable program instructions for performing the computer-implemented methods can typically be downloaded to computer 701 from an external computer or external storage device through a network adapter card or network interface included in network module 715.

WAN 702 is any wide area network (for example, the internet) capable of communicating computer data over non-local distances by any technology for communicating computer data, now known or to be developed in the future. In some embodiments, the WAN 702 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 703 is any computer system that is used and controlled by an end user (for example, a customer of an enterprise that operates computer 701) and may take any of the forms discussed above in connection with computer 701. EUD 703 typically receives helpful and useful data from the operations of computer 701. For example, in a hypothetical case where computer 701 is designed to provide a recommendation to an end user, this recommendation would typically be communicated from network module 715 of computer 701 through WAN 702 to EUD 703. In this way, EUD 703 can display, or otherwise present, the recommendation to an end user. In some embodiments, EUD 703 may be a client device, such as thin client, heavy client, mainframe computer, desktop computer and so on.

Remote server 704 is any computer system that serves at least some data and/or functionality to computer 701. Remote server 704 may be controlled and used by the same entity that operates computer 701. Remote server 704 represents the machine(s) that collect and store helpful and useful data for use by other computers, such as computer 701. For example, in a hypothetical case where computer 701 is designed and programmed to provide a recommendation based on historical data, then this historical data may be provided to computer 701 from remote database 730 of remote server 704.

Public cloud 705 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 705 is performed by the computer hardware and/or software of cloud orchestration module 741. The computing resources provided by public cloud 705 are typically implemented by virtual computing environments that run on various computers making up the computers of host physical machine set 742, which is the universe of physical computers in and/or available to public cloud 705. The virtual computing environments (VCEs) typically take the form of virtual machines from virtual machine set 743 and/or containers from container set 744. It is understood that these VCEs may be stored as images and may be transferred among and between the various physical machine hosts, either as images or after instantiation of the VCE. Cloud orchestration module 741 manages the transfer and storage of images, deploys new instantiations of VCEs and manages active instantiations of VCE deployments. Gateway 740 is the collection of computer software, hardware, and firmware that allows public cloud 705 to communicate through WAN 702.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 706 is similar to public cloud 705, except that the computing resources are only available for use by a single enterprise. While private cloud 706 is depicted as being in communication with WAN 702, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 705 and private cloud 706 are both part of a larger hybrid cloud.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment ("CPP embodiment" or “CPP”) is a term used in the present disclosure to describe any set of one, or more, storage media (also called "mediums") collectively included in a set of one, or more, storage devices that collectively include machine readable code corresponding to instructions and/or data for performing computer operations specified in a given CPP claim. A "storage device" is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits / lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of controlling VOC (Volatile Organic Compound) off-gassing in a datacenter, the method comprising:
receiving sensor data from a plurality of VOC sensors in the datacenter; and
controlling off-gassing in the datacenter based on the sensor data and human presence within the datacenter.

2. The method of claim 1, wherein controlling off-gassing in the datacenter based on the sensor data and human presence within the datacenter further comprises:
increasing off-gassing in one or more locations in the datacenter at which the sensor data indicates a level of VOC and there is no human presence within a predefined distance from the one or more locations.

3. The method of claim 1, wherein controlling off-gassing in the datacenter based on the sensor data and human presence within the datacenter further comprises:
decreasing off-gassing in one or more locations in the datacenter at which the sensor data indicates a level of VOC exceeding a predetermined threshold and there is a human present within a predefined distance from the one or more locations.

4. The method of claim 1, further comprising:
determining one or more locations of human presence within the datacenter based on maintenance schedules.

5. The method of claim 1, further comprising:
determining one or more locations of human presence within the datacenter based on real-time presence detection data.

6. The method of claim 1, further comprising:
generating a route for human traffic within the datacenter based on the sensor data.

7. The method of claim 6, further comprising:
highlighting the route within the datacenter.

8. The method of claim 1, further comprising:
highlighting locations in the datacenter based on the sensor data.

9. The method of claim 1, further comprising:
generating a representation of VOC concentrations in the datacenter based on the sensor data; and
providing the representation to a user.

10. An apparatus controlling VOC (Volatile Organic Compound) off-gassing in a datacenter, the apparatus comprising:
a processing device; and
memory operatively coupled to the processing device, wherein the memory stores computer program instructions that, when executed, cause the processing device to:
receive sensor data from a plurality of VOC sensors in the datacenter; and
control off-gassing in the datacenter based on the sensor data and human presence within the datacenter.

11. The apparatus of claim 10, wherein the computer program instructions that, when executed, cause the processing device to control off-gassing in the datacenter further comprise computer program instructions that cause the processing device to:
increase off-gassing in one or more locations in the datacenter at which the sensor data indicates a level of VOC and there is no human presence within a predefined distance from the one or more locations.

12. The apparatus of claim 10, wherein the computer program instructions that, when executed, cause the processing device to control off-gassing in the datacenter further comprise computer program instructions that cause the processing device to:
decrease off-gassing in one or more locations in the datacenter at which the sensor data indicates a level of VOC exceeding a predetermined threshold and there is a human present within a predefined distance from the one or more locations.

13. The apparatus of claim 10, further comprising instructions that, when executed, cause the processing device to:
determine one or more locations of human presence within the datacenter based on maintenance schedules.

14. The apparatus of claim 10, further comprising instructions that, when executed, cause the processing device to:
determine one or more locations of human presence within the datacenter based on real-time presence detection data.

15. The apparatus of claim 10, further comprising instructions that, when executed, cause the processing device to:
generate a route for human traffic within the datacenter based on the sensor data.

16. The apparatus of claim 15, further comprising instructions that, when executed, cause the processing device to highlight the route within the datacenter.

17. The apparatus of claim 10, further comprising instructions that, when executed, cause the processing device to highlight locations in the datacenter based on the sensor data.

18. The apparatus of claim 10, further comprising instructions that, when executed, cause the processing device to:
generate a representation of VOC concentrations in the datacenter based on the sensor data; and
provide the representation to a user.

19. A computer program product for controlling VOC (Volatile Organic Compound) off-gassing in a datacenter, the computer program product comprising a computer readable storage medium, wherein the computer readable storage medium comprises computer program instructions that, when executed:
receive sensor data from a plurality of VOC sensors in the datacenter; and
control off-gassing in the datacenter based on the sensor data and human presence within the datacenter.

20. The computer program product of claim 19, wherein the computer program instructions that control off-gassing in the datacenter further comprise computer program instructions to:
increase off-gassing in one or more locations in the datacenter at which the sensor data indicates a level of VOC and there is no human presence within a predefined distance from the one or more locations; and
decrease off-gassing in one or more locations in the datacenter at which the sensor data indicates a level of VOC exceeding a predetermined threshold and there is a human present within a predefined distance from the one or more locations.
